(19) United States
(12) Patent Application Publication
(10) Pub. No.: US 2012/0316490 A1
(43) Pub. Date: Dec. 13, 2012

(54) SURGICAL SYSTEM FOR DAMPENING VIBRATIONS BETWEEN A CANNULA AND A SURGICAL INSTRUMENT

(76) Inventor: James T. Perkins, St. Charles, MO (US)

(21) Appl. No.: 13/156,497

(22) Filed: Jun. 9, 2011

(51) Int. Cl. A61F 9/007

(52) U.S. Cl. 604/22

(57) ABSTRACT

A surgical system 10 includes a cannula 12 having a hub 14 for insertion through an incision 18 at a surgical site 20. The cannula 12 has a lumen 22 extending from a distal end 24 through the hub 14. A surgical instrument 26 is for insertion into the cannula lumen 22. Vibration dampening material 30 is formed on at least one of the cannula 12 and the surgical instrument 26. The vibration dampening material 30 reduces transmission of vibrations from the surgical instrument 26 to the surgical site 20, thus reducing unwanted heating of the surgical site 20.

### BACKGROUND

1. Field

The present invention relates to ophthalmic surgery using a surgical instrument inserted through a cannula, and more specifically to dampening vibrations caused by the surgical instrument.

2. Description of Related Art

This section provides background information related to the present disclosure which is not necessarily prior art.

The present trend in ophthalmic surgery is towards smaller incisions that cause less trauma to a surgical site and require few, if any, sutures to close the incision. For example, vitreal retinal surgery has aggressively adopted surturless transconjunctival surgery using cannulas, sometimes referred to as entry site alignment devices. Such cannulas are available from manufacturers, including Bausch & Lomb Incorporated.

These cannulas are inserted, with a trocar extending through the cannula, into the posterior of the eye and provides a passage into the eye for various instruments. These instruments are typically small enough in diameter (e.g. 23 or 25 gauge) that when the cannula is removed the incision is self-sealing and no sutures are required.

It is highly undesirable for the surgical instruments to cause heat build-up at the surgical site. Excessive heat can permanently damage ocular tissue. With the use of powered surgical instruments, such as fragmentation devices, vitreous cutters, and scissors, there is potential for heat build-up due to vibration between the close fitting cannula and surgical instrument. Therefore, it would be desirable to provide a system that dampens any vibrations from the surgical instrument, such that the vibrations do not cause excessive heat build-up at the surgical site.

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

The drawings described herein are for illustrative purposes only of selected embodiments and not all possible implementations, and are not intended to limit the scope of the present disclosure.

FIG. 1 is a cross-sectional view of an eye with a system, in accordance with a preferred embodiment inserted into the eye;

FIG. 2 is a side elevation of a fragmentation needle, in accordance with a preferred embodiment;

FIG. 3 is a cross-section of FIG. 2, along line 3-3;

FIG. 4 is a side elevation of a cannula, in accordance with a preferred embodiment;

FIG. 5 is a cross-section of FIG. 4, along line 5-5;

FIG. 6 is a top view of FIG. 4;

FIG. 7 is a perspective of a vitreous cutter, in accordance with a preferred embodiment;

FIG. 8 is a partial elevation of a scissors, in accordance with a preferred embodiment; and

FIG. 9 is a partial cross-section of another embodiment.

Corresponding reference numerals indicate corresponding parts throughout the several views of the drawings.

### DETAILED DESCRIPTION OF CERTAIN PREFERRED EMBODIMENTS

Example embodiments will now be described more fully with reference to the accompanying drawings.

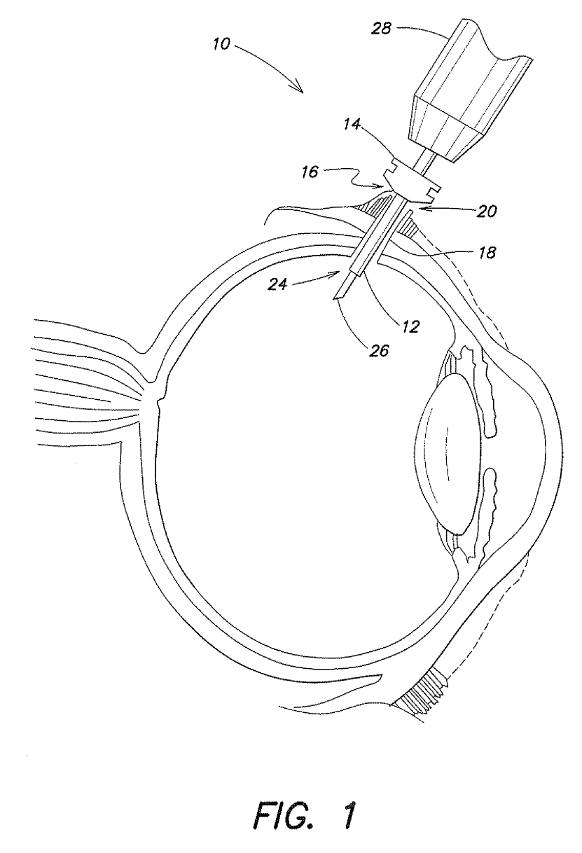

FIG. 1 is an embodiment of a surgical system 10 inserted into an eye, in accordance with the present invention. System 10 includes a cannula 12 having a hub 14 formed on a proximal end 16 and for insertion through an incision 18 at a surgical site 20. The cannula 12 has a lumen (not shown in FIG. 1) extending from a distal end 24 and through the hub 14. A surgical instrument 26 is for insertion into the cannula lumen. Vibration dampening material (shown and described in detail below) is formed on at least one of the cannula 12 and the surgical instrument 26, such that the vibration dampening material reduces transmission of vibrations from the surgical instrument 26 to the surgical site 20, thus reducing unwanted heating of the surgical site 20. In the embodiment of FIG. 1, surgical instrument 26 is a fragmentation needle that is attached to and vibrated by an ultrasonic handpiece 28, as is well known.

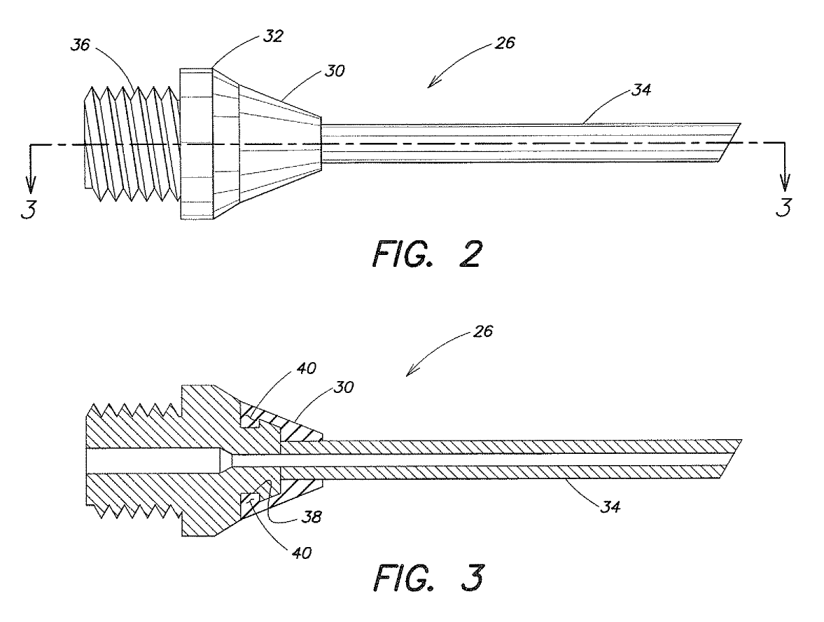

FIGS. 2 and 3 disclose a surgical instrument, such as the fragmentation needle 26 with vibration dampening material 30 formed around a hub 32 of an elongated hollow needle 34. Hub 32 may also include threads 36 for attachment to a handpiece. FIG. 3 shows one possible way for attaching vibration dampening material 30 to the fragmentation needle 26. As seen, vibration dampening material 30 is formed in a recess 38 of the hub via protrusion 40. This arrangement is easily assembled and the mating structures of the recess 38 and protrusion 40 holds the vibration dampening material 30 on the hub 32, and prevents dislocation during use. Vibration dampening material may be any material suitable for surgery that also effectively absorbs or dampens vibrations from the needle 26; such materials may include silicone, rubber, synthetic rubber, neoprene, or other suitable materials as may be known. Other than vibration dampening material 30, fragmentation needle 26 may be of standard construction and formed of standard materials, such as titanium, steel, ceramic, plastic, or other material. Vibration dampening material 30 surrounding hub 32 minimizes transmission of ultrasonic vibrations from fragmentation needle 26 to the surgical site. Minimizing transmission of vibration to the surgical site results in reduce heating of the surgical site. Vibration dampening material 30 surrounding hub 32 minimizes transmission of ultrasonic vibrations from the needle 26 to the surgical site or cannula 12. Minimizing transmission of ultrasonic vibration to the surgical site and cannula hub 14 reduces heating the surgical site or cannula 12 when the needle hub 32 is pushed against the cannula 12. The needle hub 32 contacting the cannula hub 14 may be inadvertent or may be intentional, if the surgeon needs to use the full length of the ultrasonic needle 26.

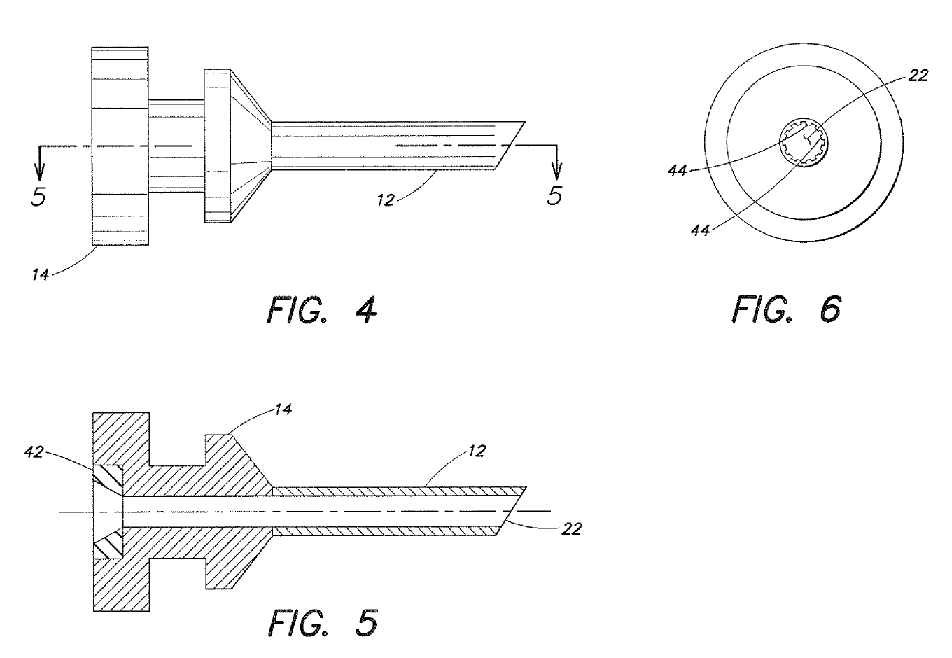

FIGS. 4 and 5 show the cannula 12 with hub 14, in accordance with an embodiment of the present invention. FIG. 5 shows vibration dampening material 42 that is the same as vibration dampening material 30, except that it is formed as a ring in hub 14. Vibration dampening material 42 may be tapered towards the lumen 22, as shown, for aiding insertion of the surgical instrument into the lumen 22. Grooves or recesses 44 may also be formed along lumen 22, as shown in FIG. 5 and best seen in FIG. 6. Grooves 44 allow fluid to flow between the cannula 12 and a surgical instrument in lumen 22. Cannula 12 may be formed of any suitable known materials, such as polyimide, steel, titanium, plastic, or other material. Cannula 12 may be of varying cross sectional dimension as well as having varying lumen 22 cross sectional dimension.

The system 10 may include a cannula or a surgical instrument having vibration dampening material formed on only one of the cannula or the surgical instrument, or the system could include vibration dampening material on both the cannula and the surgical instrument.

The vibration dampening material may be formed on the cannula and the surgical instrument by any acceptable method, such as adhesive, frictional contact, mating structures, coating, or other methods.

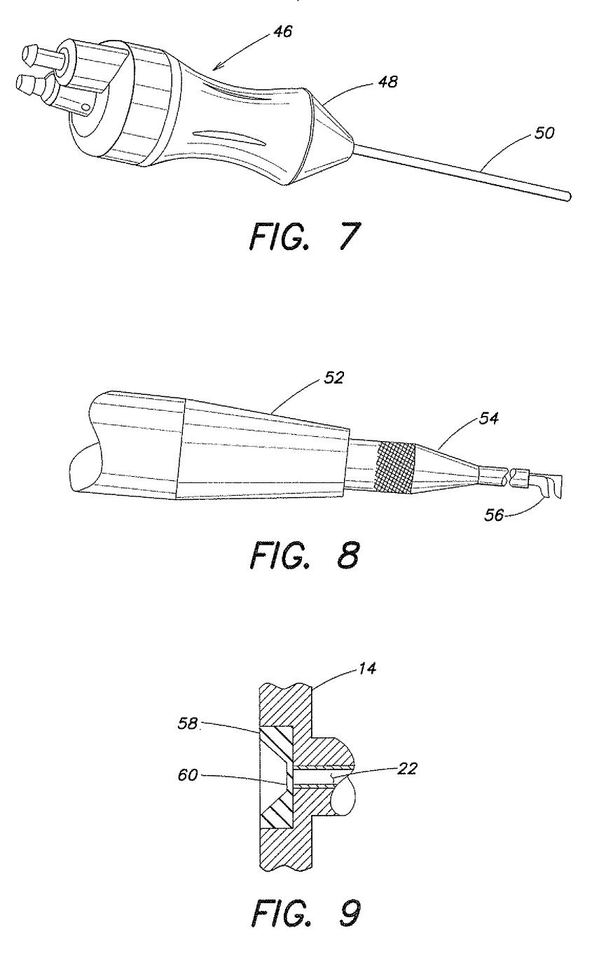

FIG. 7 discloses a vitreous cutter 46 having vibration dampening material 48 formed on a portion adjacent the cutter tube 50 for reducing the transmission of vibrations as described above.

FIG. 8 discloses a partial view of a pneumatic scissors 52 with vibration dampening material 54 attached to the scissors mechanism 56. The vibration dampening material 54 may be formed of the same materials and performs the same function as described above. Other surgical instruments, such as other needles, aspirators, tissue manipulators, morcellators, and other surgical instruments may also benefit from the addition of vibration dampening material as taught by this specification.

FIG. 9 is a partial cross section of a cannula hub 14 and is the same as that described above relative to FIGS. 4 and 5, except that vibration dampening material 58 includes a web portion 60 spanning the lumen 22. Web 60 provides a sealing function preventing leakage of fluids through lumen 22 when surgical instruments are removed. Web 60 can be punctured by a surgical instrument upon insertion as is known, or web 60 may include various slits or cuts, in a variety of geometries, as is also known.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

I claim:

1. A surgical system comprising:
a cannula having a hub formed on a proximal end and for insertion through an incision at a surgical site, the cannula having a lumen extending from a distal end and through the hub;
a surgical instrument for insertion into the cannula lumen; and
vibration dampening material formed on at least one of the cannula and the surgical instrument, such that the vibration dampening material reduces transmission of vibrations from the surgical instrument to the surgical site and thereby, reducing unwanted heating of the surgical site.

2. The system of claim 1 wherein the vibration dampening material is formed in a recess of the hub.

3. The system of claim 2 wherein the vibration dampening material is tapered towards the lumen for aiding insertion of the surgical instrument into the lumen.

4. The system of claim 1 wherein the surgical instrument is a fragmentation needle and the vibration dampening material is formed around a hub of the fragmentation needle.

5. The system of claim 1 wherein the vibration dampening material is formed on each of the cannula and the surgical instrument.

6. The system of claim 1 wherein the surgical instrument is one of a vitreous cutter and a scissors.

7. The system of claim 1 wherein the vibration dampening material is adhered to the cannula or the surgical instrument.

8. The system of claim 1 wherein the vibration dampening material is held on the cannula or surgical instrument through frictional contact.

9. The system of claim 1 wherein the vibration dampening material and at least one of the cannula and the surgical instrument have mating structures for holding the vibration dampening material on the cannula or the surgical instrument.

10. The system of claim 1 wherein the cannula further includes at least one groove formed along the lumen.

11. The system of claim 2 wherein the vibration dampening material forms a web spanning the lumen.
